(19) United States
(12) Patent Application Publication
(10) Pub. No.: US 2026/0284120 A1
(43) Pub. Date: Sep. 24, 2026

(54) Methods And Device for Processing Biological Material

(71) Applicant: Push2Spin Technologies, Inc., Pittsburgh, PA (US)

(72) Inventors: Jeffrey A Gusenoff, Pittsburgh, PA (US); Beth R Gusenoff, Pittsburgh, PA (US); Denise Gottfried, Woodside, CA (US)

(73) Assignee: Push2Spin Technologies, Inc., Pittsburgh, PA (US)

(21) Appl. No.: 19/571,178

(22) Filed: Mar. 18, 2026

Related U.S. Application Data
(60) Provisional application No. 63/773,976, filed on Mar. 18, 2025.

(51) Int. Cl. A61K 35/35; A61J 1/16; A61J 1/20

(52) U.S. Cl. CPC A61K 35/35; A61J 1/16; A61J 1/2086

(57) ABSTRACT

A biological tissue processing device may include a first syringe configured to contain biological material. A second syringe may be fluidly connectable to the first syringe such that biological material can be transferred from the first syringe to the second syringe. A processing module may be fluidly coupled to the first syringe and the second syringe. The processing module may include an aperture such that biological material passes through the aperture as the biological material moves between the first syringe and the second syringe. An actuator may be configured to rotate at least one of the first syringe, the second syringe, or the processing module. Rotation of the first syringe, the second syringe, or the processing module may generate centrifugal force that separates components of the biological material.

### CROSS-REFERENCE TO RELATED APPLICATIONS

This application claims the benefit of U.S. Provisional Patent Application No. 63/773,976 filed Mar. 18, 2025 entitled “Methods And Device for Micronizing, Centrifuging, and Purification of Adipose Tissue”, which is incorporated by reference herein in its entirety.

### FIELD OF THE INVENTION

The present disclosure relates generally to devices for processing biological material. More particularly, the present disclosure relates to systems and devices for processing adipose tissue, including systems configured to micronize adipose tissue and separate adipose tissue from oil and other biological components prior to reinjection into a patient.

### BACKGROUND OF THE INVENTION

Adipose tissue is frequently harvested from a patient and subsequently processed for reinjection in a variety of medical and cosmetic procedures. The adipose tissue may be harvested using liposuction techniques and later reinjected to provide volume restoration, tissue regeneration, or aesthetic enhancement. In many cases, the harvested adipose tissue is processed before reinjection to improve the quality and consistency of the injected material. Processing of harvested adipose tissue may include separating adipose tissue from other biological components such as blood, oil, or other fluids that may be present in the harvested material.

Various devices and techniques have been developed to assist in processing adipose tissue. However, such devices and techniques require removing the tissue from the surgical room for processing at a remote location. This increases the chance of infection during a surgical procedure. Other devices are cumbersome to use or do not sufficiently process the biological material. Accordingly, there remains a need for improved systems and devices for processing biological material such as adipose tissue prior to injection into a patient.

### BRIEF SUMMARY OF THE INVENTION

A biological tissue processing device may include a first syringe configured to contain biological material, a second syringe fluidly connectable to the first syringe to receive biological material from the first syringe, a processing module fluidly coupled to the first syringe and the second syringe and an actuator. The processing module may include an aperture such that biological material passes through the aperture as the biological material passes between the first syringe and the second syringe. The actuator may be configured to rotate at least one of the first syringe, the second syringe, and the processing module to generate centrifugal force that separates components of the biological material.

The actuator may rotate the first syringe and the second syringe simultaneously. The actuator may rotate the first syringe, the second syringe, and the processing module simultaneously. The actuator may rotate the first and second syringes about a common axis. The actuator may comprise a manually activated device that converts linear motion to rotational motion. The actuator and may be detachably coupled to the first syringe.

The processing module may include a plurality of transfer arms extending outward from the central hub with each transfer arm including at least one aperture. The aperture may be one of a plurality of apertures. The transfer arms may be circumferentially spaced about the central hub. The aperture may be one of a plurality of apertures and each aperture may mechanically reduce a particle size of the biological material passing through the connector. The apertures may have progressively decreasing diameters. The processing module may include a filter, and the processing module may further comprise an absorbent material configured to absorb oil separated from adipose tissue. The aperture may be one of a plurality of apertures and each of the first and second syringes may be detachably coupled to the same aperture when biological material passes from the first syringe to the second syringe.

A biological tissue processing device may include a base, a frame extending from the base and an actuator coupled to the frame. The frame may be configured to hold a syringe containing biological material. The actuator may be configured to engage and rotate the syringe to separate adipose tissue from oil or other biological components. The device may further include a first syringe configured to contain adipose tissue, a second syringe fluidly connectable to the first syringe to receive biological material from the first syringe, and a processing module fluidly coupled to the first syringe and the second syringe.

The first syringe may be coupleable to the frame. The second syringe may be coupleable to the base. The processing module may include an aperture such that biological material passes through the aperture as the biological material passes between the first syringe and the second syringe. The actuator may include a push button, wherein linear motion of the push button is converted into rotational motion of the syringe.

The device may further include an arm movably coupled to the frame. The arm may be configured to move the push button linearly to cause rotational motion of the syringe. The device may include a motor coupled to the arm to move the arm relative to the frame. The frame and the base may define a receiving area for the syringe. At least a portion of the frame may be moveable relative to the base from a first position to a second position. The receiving area may have a first height when the frame is in the first position and a second height different from the first height when the frame is in the second position. At least one of the frame and the base may include a locking mechanism configured to hold the syringe in a fixed position during actuation. The base may include a recess to receive at least a portion of the syringe to stabilize the syringe device during operation.

A method of processing adipose tissue may include placing adipose tissue into a first syringe, connecting the first syringe to a processing module including an aperture, connecting a second syringe to the processing module, transferring the adipose tissue from the first syringe through the aperture and to the second syringe, and rotating at least one of the processing module, the first syringe, and the second syringe to generate centrifugal force that separates adipose tissue from oil or other biological components. The aperture may be one of a plurality of apertures and the method may further comprise sequentially passing the adipose tissue through each of the plurality of apertures to micronize the adipose tissue. The first syringe may be coupled to an actuator and the method may include manually activating the actuator to rotate at least one of the processing module, the first syringe, and the second syringe. The rotating step may include simultaneously rotating each of the processing module, the first syringe, and the second syringe about a common axis. The method may further comprise reinjecting processed adipose tissue into a patient.

### BRIEF DESCRIPTION OF THE SEVERAL VIEWS OF THE DRAWINGS

The foregoing summary, as well as the following detailed description of embodiments of the processing assembly, will be better understood when read in conjunction with the appended drawings of an exemplary embodiment. It should be understood, however, that the invention is not limited to the precise arrangements and instrumentalities shown.

In the drawings:

FIG. 1 is a front elevation view of a processing assembly in accordance with an exemplary embodiment of the present disclosure;

FIG. 2 is a front elevation view of the syringe of FIG. 1;

FIG. 3 is a top plan view of the processing module of FIG. 1;

FIG. 4 is a top plan view of another processing module in accordance with an exemplary embodiment of the present disclosure;

FIG. 5 is a top plan view of another processing module in accordance with an exemplary embodiment of the present disclosure;

FIG. 6 is a front elevation view of another embodiment of an actuator and a syringe barrel in accordance with an exemplary embodiment of the present disclosure;

FIG. 7 is a front elevation view of another embodiment of an actuator and a plunger in accordance with an exemplary embodiment of the present disclosure;

FIG. 8 is a front elevation view of the actuator of FIG. 6 with the processing module and syringe of FIG. 1;

FIG. 9 is a front elevation view of the actuator and syringe of FIG. 8;

FIG. 10 is a top perspective view of a support structure and a processing assembly in accordance with an exemplary embodiment of the present disclosure;

FIG. 11 is a side elevation view of a support structure and processing assembly in accordance with another exemplary embodiment of the present disclosure;

FIG. 12 is a side elevation view of the support structure, actuator, and syringe of FIG. 11;

FIG. 13 is a side elevation view of a support structure, actuator, and syringe in accordance with another exemplary embodiment of the present disclosure; and

FIG. 14 is a side elevation view of a support structure, actuator, automated actuation assembly, and syringe in accordance with another exemplary embodiment of the present disclosure.

### DETAILED DESCRIPTION OF THE EMBODIMENTS OF THE INVENTION

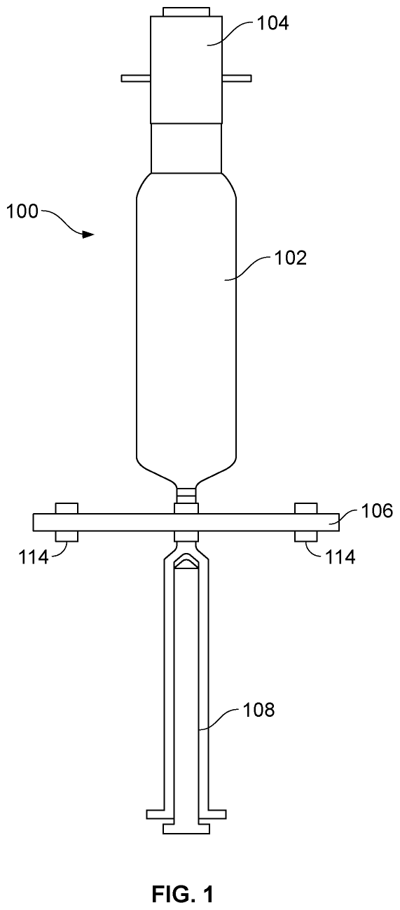

Referring to the drawings in detail, wherein like reference numerals indicate like elements throughout the several views, FIG. 1 illustrates a biological tissue processing assembly, generally designated 100, in accordance with an exemplary example of the present disclosure. The processing assembly 100 may be configured to process biological materials and separate components thereof. In some examples, the processing assembly 100 may be configured to separate adipose tissue from oil and other biological components. For example, the processing assembly 100 may be used to process adipose tissue harvested from a patient using liposuction or other medical procedures. In some examples, the processing assembly 100 may mechanically micronize the adipose tissue to reduce particle size, and may thereafter separate adipose tissue from oil or other biological fluids using centrifugal forces. The processed adipose tissue may then be delivered to a syringe. The processed adipose tissue may be further processed in the syringe and then reinjected into a patient for therapeutic or cosmetic procedures.

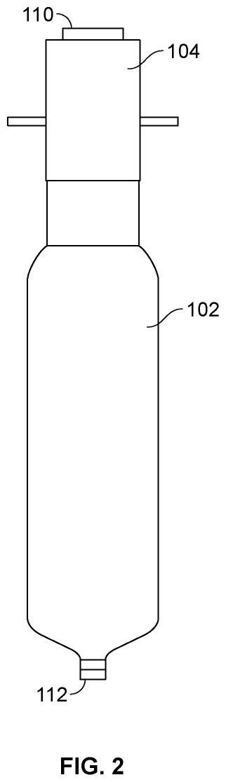

The processing assembly 100 may include a container configured to receive biological material. In some examples, the container may be a syringe 102. The syringe 102 may include a cylindrical barrel defining an internal chamber configured to receive biological material and a movable plunger disposed within the barrel. The plunger may be movable relative to the barrel to draw biological material into the syringe 102 or expel biological material from the syringe 102. The syringe 102 may further include a distal outlet configured to fluidly communicate with other components of the processing assembly 100. The outlet may include a coupling element 112 (FIG. 2) configured to detachably couple the syringe 102 with additional devices or modules. The coupling element 112 may be a Luer connector such as a Luer lock connector.

In some examples, the processing assembly 100 may further include a processing module 106 configured to process biological material passing through the device. The processing module 106 may be fluidly coupled to the syringe 102. The processing module 106 may be configured to permit biological material to pass from the syringe 102 through the processing module 106 and into a second syringe 108. The biological material may be transferred between the first and second syringes 102 and 108 multiple times to progressively micronize the adipose tissue. The second syringe 108 may be fluidly coupled to the processing module 106 through a connector such as a Luer connector. The second syringe 108 may receive processed biological material, such as micronized adipose tissue, which may subsequently be at least one of further processed and injected into a patient.

The processing assembly 100 may further include an actuator 104 configured to actuate the syringe 102 and cause rotation of at least a portion of the processing assembly 100. In some examples, the actuator 104 may be configured to rotate the syringe 102 about a rotational axis extending generally along a longitudinal axis of the syringe. Rotation of the syringe 102 may generate centrifugal forces within the syringe that promote separation of adipose tissue from oil or other biological components. The syringe 102, processing module 106, and any associated actuator 104 may be arranged along a common rotational axis. Rotation of one component may therefore cause rotation of the remaining components when the components are rotationally coupled. This arrangement allows centrifugal forces to be generated within the biological material.

The actuator 104 may further be configured to actuate the syringe plunger to cause biological material to move through the processing module 106. For example, actuation of the syringe plunger may cause biological material contained within the syringe 102 to be expelled from the syringe 102, through the processing module 106, and into the second syringe 108.

The actuator 104 may comprise a push-to-spin device. Referring to FIG. 2, the actuator 104 may include an activator 110 configured to receive manual or automated input. In some examples, the activator 110 may comprise a push button that is movable along a generally linear path. The actuator 104 may include a conversion mechanism configured to convert linear movement of the activator 110 into rotational movement of the syringe 102. For example, the conversion mechanism may include one or more mechanical elements such as cams, ratchet mechanisms, gears, or similar motion-conversion components that translate linear motion of the push button into rotational motion of the syringe.

The actuator 104 may comprise a powered device configured to generate motion for rotating the syringe or moving a plunger within the syringe. For example, the actuator 104 may include an electric motor, servo motor, stepper motor, or other powered drive mechanism capable of generating rotational or linear motion. The actuator 104 may include an internal power source such as one or more batteries. The batteries may be rechargeable or disposable. In other configurations, the actuator 104 may be configured to receive power from an external source, such as a wall outlet, medical equipment power supply, or other external power system. The actuator 104 may therefore include a power interface, such as a power cord, connector, or charging port, configured to supply electrical power to the actuator.

The actuator 104 may further include control circuitry configured to regulate operation of the powered device. For example, the actuator may include one or more switches, buttons, or electronic controls configured to allow a user to start, stop, or adjust operation of the actuator. The actuator may also include speed control components configured to vary the rotational speed of the syringe or plunger during operation.

Repeated activation of the activator 110 may cause repeated rotation of the syringe 102 about the rotational axis. In some examples, repeated activation of the activator 110 may cause the syringe 102 to spin at a sufficient rotational speed to generate centrifugal forces required to separate adipose tissue from oil or other biological components contained within the syringe 102. For example, adipose tissue may migrate toward one region of the assembly while oil, blood, or other biological fluids may migrate toward another region of the assembly.

In some examples, the actuator 104 and the syringe 102 may form a unified device. For example, the actuator 104 may be integrated with the syringe 102 to form a combined syringe and push-to-spin processing assembly. In other examples, the actuator 104 may be detachably coupled to the syringe 102. For example, the actuator 104 may include a coupling interface configured to engage the coupling element 112 of the syringe 102. In some examples, the coupling interface may comprise a Luer connector, threaded connector, snap-fit connector, or other detachable coupling mechanism.

In some examples, the syringe 102 and actuator 104 may correspond to a syringe processing device such as that described in U.S. Pat. No. 11,918,793, the entire disclosure of which is incorporated herein by reference. However, it should be appreciated that other syringe configurations and actuation mechanisms may also be used. The actuator 104 may therefore be configured to operate with a variety of syringe types and sizes.

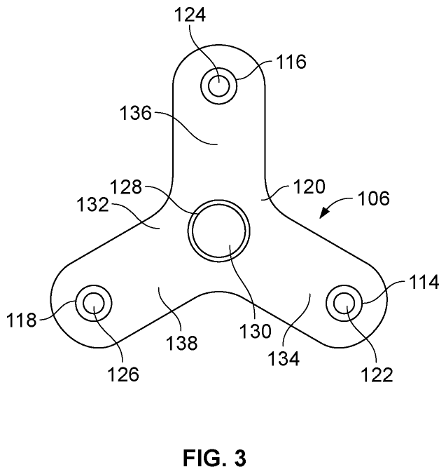

Referring to FIG. 3, the processing module 106 may be configured to mechanically micronize biological material passing therethrough. In some examples, the processing module 106 may include one or more micronizing elements, transfer connectors, filters, meshes, or sizing elements configured to mechanically reduce the particle size of adipose tissue or other biological material. The processing module 106 may be provided as a disposable cartridge configured for single use.

The processing module 106 may include a body 120 having a generally planar or disk-like configuration. The body 120 may include a first surface 128 and a second surface opposite the first surface 128. The processing module 106 may further include a plurality of transfer connectors extending from the body 120. In the illustrated example, the processing module 106 includes a first transfer connector 114, a second transfer connector 116, and a third transfer connector 118.

The plurality of transfer connectors 114, 116, and 118 may define coupling mechanisms configured to fluidly couple the processing module 106 to the syringe 102 and the second syringe 108. In some examples, the transfer connectors 114, 116, and 118 may comprise Luer connectors, such as female Luer lock connectors. The transfer connectors 114, 116, and 118 may extend away from the first surface 128 and/or the second surface of the body 120 so that the connectors can fluidly couple to the first and second syringes 102 and 108. Alternatively, the transfer connectors 114, 116, and 118 may be recesses adapted to receive a portion of the first or second syringe 102 and 108.

The transfer connectors 114, 116, and 118 may define a first aperture 122, a second aperture 124, and a third aperture 126, respectively. Each of the apertures 122, 124, and 126 may extend through the body 120 such that biological material can pass through the apertures and therefore through the processing module 106. Each aperture may extend along an aperture central axis. The central axes of the apertures 122, 124, and 126 may be parallel.

The apertures 122, 124, and 126 may have different sizes to facilitate sequential micronization of the biological material. The first aperture 122 may be larger than the second and third apertures 124 and 126. For example, the first aperture 122 may have a diameter larger than the diameters of the second and third apertures 124 and 126. The second aperture 124 may have a diameter that is smaller than the diameter of the first aperture 122 but larger than the diameter of the third aperture 126. The third aperture 126 may have a diameter smaller than the diameters of the first and second apertures 122 and 124. The apertures may have diameters between approximately 0.1 mm and 3 mm. The apertures may have diameters of 2.4 mm, 1.4 mm, and 1.2 mm. The diameter of the apertures may be smaller than a diameter of an outlet of one or both of the first and second syringes 102 and 108.

The biological material may be transferred between the first syringe 102 and the second syringe 108 through one or more of the apertures 122, 124, and 126 of the processing module. For example, biological material may be expelled from the first syringe 102 and passed through the first aperture 122 into the second syringe 108. The biological material may then be transferred from the second syringe 108 back into the first syringe 102 through the second aperture 124. In some implementations, the biological material may subsequently be transferred through the third aperture 126. Each passage through the apertures may reduce the size of adipose tissue particles contained within the biological material. As a result, repeated transfers of biological material between the syringes through the processing module 106 may progressively micronize the adipose tissue to produce micronized fat or nanofat suitable for reinjection. In some configurations, the first syringe 102, the processing module 106, and the second syringe 108 may remain fluidly connected during processing such that biological material can be repeatedly transferred between the syringes through the processing module in a closed fluid pathway without exposure to the external environment.

The biological material may be transferred between the syringes 102 and 108 multiple times during processing. The biological material may also pass through any one of the apertures 122, 124, and 126 more than once.

The processing module 106 may further include a hub 128 positioned on a central portion 132 of the processing module 106. The hub 128 may define a coupling mechanism configured to fluidly couple the first syringe 102 and the second syringe 108 to the processing module 106. The hub 128 may include an opening 130 extending through the body 120 to fluidly couple the first syringe 102 and the second syringe 108 to each other.

In some examples, the opening 130 may have a larger diameter than the apertures 122, 124, and 126 to facilitate efficient transfer of biological material between the syringes. In other examples, the processing module 106 may include the hub 128 without the opening 130, and biological material may instead be transferred through one or more of the apertures 122, 124, or 126.

At least one of the first and second syringes 102 and 108 may be rotated when the syringes are coupled to the hub 128. Rotation of the syringes 102 and 108 may generate centrifugal forces within the syringes that promote separation of adipose tissue from oil or other biological components. The rotation may also further assist in micronizing the biological material by causing agitation or mixing of the biological material during processing.

The hub 128 may be positioned on the central portion 132 of the processing module 106. The processing module 106 may include a plurality of arms extending outward from the central portion 132. In the illustrated example, the plurality of arms includes a first arm 134, a second arm 136, and a third arm 138. The number of arms may vary and may include two arms, three arms, four arms, or more. The first transfer connector 114 may be positioned on the first arm 134, the second transfer connector 116 may be positioned on the second arm 136, and the third transfer connector 118 may be positioned on the third arm 138. The hub 128 may define a rotational axis of the processing module 106 such that the arms 132, 134, and 136 extend outward from the hub 128 while remaining balanced about the rotational axis during rotation of the processing module 106.

The plurality of arms may be circumferentially spaced around the central portion 132 of the processing module 106. In some examples, the arms 134, 136, and 138 may be spaced approximately equally around the central portion 132 such that the processing module 106 has a generally symmetric configuration. The arms 134, 136, and 138 may be spaced approximately 120 degrees apart such that the processing module forms a generally star-shaped or tri-lobed structure. In some examples, the arms may have approximately the same length such that the transfer connectors are positioned at similar radial distances from the central portion 132.

The syringe 102 may be rotationally fixed relative to the processing module 106 when the coupling element 112 engages the hub 128. In such examples, rotational movement applied to the syringe 102 may be transmitted to the processing module 106 so that the processing module 106 rotates together with the syringe 102 about a common rotational axis. The second syringe 108 may be rotationally fixed relative to the processing module 106 when the second syringe 108 is coupled to the hub 128. In such examples, the second syringe 108 may rotate as the processing module 106 rotates.

In some examples, the second syringe 108 may also be rotationally fixed relative to the processing module 106 when the second syringe 108 is coupled to the hub 128. For example, the second syringe 108 may include a connector configured to engage the hub 128 in a manner that prevents relative rotation between the second syringe 108 and the processing module 106. In such examples, the second syringe 108 may rotate together with the processing module 106 and the first syringe 102 during operation of the processing assembly 100.

Accordingly, in some examples, the first syringe 102, the processing module 106, and the second syringe 108 may form a rotationally coupled assembly that rotates together about the rotational axis. Rotation of the assembly may generate centrifugal forces within the syringes that facilitate separation of adipose tissue from oil or other biological components contained within the biological material. In some examples, the rotation may also promote mixing or agitation of the biological material as the biological material passes between the syringes through the processing module 106.

One or more of the syringes 102 and 108 may be permitted to rotate independently of the processing module 106. For example, the hub 128 may include one or more bearings, bushings, or other rotational interfaces configured to permit relative rotation between the syringes and the processing module 106. Such configurations may allow rotation of the processing module 106 while one or both syringes remain stationary relative to other portions of the processing assembly 100.

The first syringe 102, the processing module 106, and the second syringe 108 may be arranged along a common rotational axis. The rotational axis may extend generally along the longitudinal axis of the syringes 102 and 108 and through the central portion 132 of the processing module 106. When the first syringe 102 is actuated by the actuator 104, rotational movement may be transmitted through the coupling element 112 and the hub 128 so that the first syringe 102, the processing module 106, and the second syringe 108 rotate together as a unit about the common rotational axis. In some examples, the central portion 132 and hub 128 may function as a rotational interface that aligns the syringes along the rotational axis and transmits rotational motion between the components. The aligned configuration of the syringes and the processing module may allow the biological material to remain within a closed fluid path while the entire syringe-module-syringe assembly rotates during operation. Rotation of the syringe-processing module-syringe assembly about the common rotational axis may generate centrifugal forces within the biological material contained in the syringes. The centrifugal forces may cause components having different densities to separate within the biological material. For example, adipose tissue may separate from oil or other biological fluids during rotation.

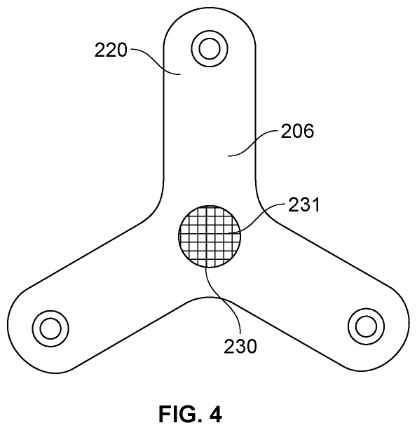

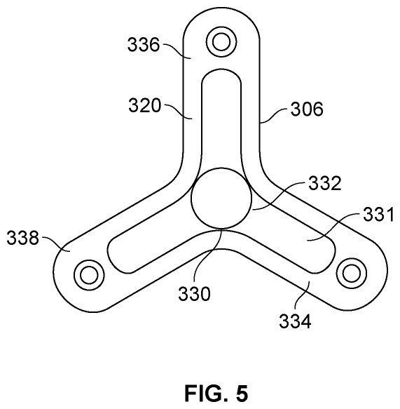

Micronized tissue may be transferred from the syringe 102 to the second syringe 108. The syringe 102 may then be decoupled from at least one of the processing module 106 and the second syringe 108. The actuator 104 may then be coupled with the second syringe 108 as detailed below with reference to FIGS. 4 and 5. The actuator 104 may rotate the second syringe 108 to further separate fat and oil components of the micronized tissue. The further separated tissue may then be injected into a patient or returned to syringe 102 for further processing.

Referring now to FIGS. 4 and 5, alternative constructions of a processing module 206 and 306 are shown. It should be appreciated that the processing modules 206 and 306 are similar to processing module 106 and therefore only the differences between the processing modules 206 and 306 are discussed in detail herein. Similar components are identified using similar reference numerals in the “200” and “300” series.

The processing module 206 may include a filter 231 positioned in the opening 230. In some examples, the filter 231 may be configured to further micronize biological material passing through the processing module 206. The filter 231 may comprise a micronizing filter or a mesh filter. The filter 231 may be positioned between the first and second surfaces of the body 220. Biological material passing through the processing module 206 may therefore pass through the filter 231 before reaching the second syringe.

The processing module 306 may include an absorbent material 331 positioned adjacent the opening 330. In some examples, the absorbent material 331 may be configured to absorb oil separated from adipose tissue. The processing module 306 may include a track, channel, or recess in which the absorbent material 331 is positioned. The track may extend from the central portion 332 along at least one of the plurality of arms 334, 336, and 338. During operation, oil separated from the biological material may be absorbed by the absorbent material 331, thereby improving the purity of the processed adipose tissue.

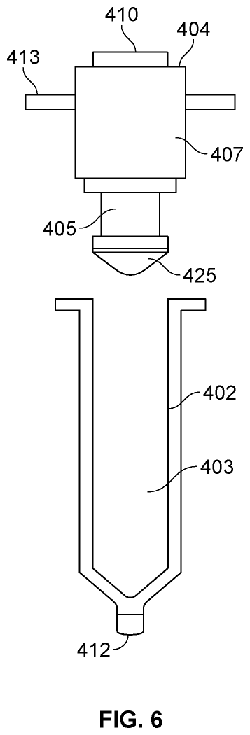

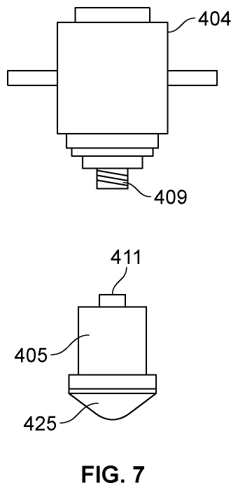

Referring to FIGS. 6 and 7, alternative constructions of a syringe 402 and actuators 404 are shown. It should be appreciated that the syringe 402 and actuator 404 are similar to the syringe 102 and actuator 104 and only the differences between the devices are discussed in detail herein. Similar components are identified using similar reference numerals in the “400” series.

The syringe 402 may include a barrel 403 configured to receive biological material. In some examples, the barrel 403 may have a generally cylindrical shape defining an internal chamber configured to receive adipose tissue or other biological materials. The barrel 403 may include a distal outlet that is fluidly connected to a coupling element 412. The coupling element 412 may comprise a Luer connector, such as a Luer lock connector, configured to detachably couple the syringe 402 with other components of the processing assembly, including the processing module 106 described above. In some examples, biological material contained within the barrel 403 may be expelled from the barrel through the coupling element 412 when a plunger is advanced within the barrel.

The actuator 404 may be detachably coupleable with the syringe 402. The actuator 404 may include a plunger 405 sized and shaped to be positioned in the barrel 403. A sidewall of the barrel 403 and the plunger 405 may form a fluid-tight seal when the plunger 405 is positioned within the barrel 403. The plunger 405 may be manufactured from rubber, elastomeric material, silicone, or another resilient material capable of forming a fluid seal while maintaining compatibility with biological materials contained within the syringe. The engagement between the plunger 405 and the barrel 403 may also rotationally fix the plunger relative to the barrel. In such examples, rotation of the plunger 405 may cause the barrel 403 to rotate together with the plunger.

The actuator 404 may be detachably coupleable with a plurality of plungers 405 having different lengths and diameters to accommodate syringes of different sizes and configurations. For example, the actuator 404 may be configured for use with syringes having volumes between approximately 1 mL and 60 mL, although larger or smaller syringes may also be utilized. The plunger 405 may include an exchangeable plunger tip 425 configured to interface with different syringe sizes, geometries, or plunger head configurations. The plunger tip 425 may include one or more engagement features such as a Leur lock, threads, press-fit features, snap-fit features, or keyed interfaces to facilitate secure coupling with a corresponding syringe plunger. The plunger tip 425 may include a coating, such as a silicone coating or other lubricating coating, to facilitate smooth insertion and movement within the barrel 403 and to reduce friction during operation. In some configurations, the plunger 405 and plunger tip 425 may be formed from materials selected to minimize interaction with biological materials and maintain sealing performance over repeated use.

Components of the actuator 404, the plunger 405, the plunger tip 425, and the syringe 402 may be provided in sterile or non-sterile configurations depending on the intended application. In some examples, one or more components may be disposable and intended for single use. One or more components may be configured for repeated use and may be constructed from materials capable of withstanding sterilization processes. Suitable sterilization processes may include, for example, autoclave sterilization, ethylene oxide sterilization, or other sterilization techniques.

The actuator 404 may include a drive assembly configured to move the plunger 405 relative to the barrel 403. The drive assembly may be configured to rotate the plunger 405, move the plunger 405 linearly within the barrel 403, or provide a combination of linear and rotational movement. Linear movement of the plunger 405 may expel biological material from the syringe 402, while rotational movement may cause the syringe 402 to rotate about a rotational axis extending along the longitudinal axis of the barrel 403. The drive assembly may be positioned within a housing 407 of the actuator 404.

The actuator 404 may include an activator 410 configured to allow a user to manually power the actuator. For example, the activator 410 may comprise a push button or similar control element that, when actuated, causes the drive assembly to rotate or advance the plunger 405. The drive assembly may include elements that form a drive train configured to transmit motion from the activator 410 to the plunger 405 or ram 409. The drive train may include one or more gears, gear trains, pinion gears, racks, threaded shafts, lead screws, cams, ratchets, pawls, or other motion conversion mechanisms. The drive train may convert linear motion of the activator 410 into rotational motion of the plunger 405 or syringe 402. The drive train may include a ratchet mechanism configured to permit incremental rotational movement of the syringe when the activator 410 is repeatedly actuated. The drive train may alternatively include a cam mechanism or gear mechanism configured to convert linear displacement of the activator 410 into continuous or stepped rotational motion.

The drive train may further include one or more biasing elements such as springs configured to return the activator 410 toward an initial position after actuation. Bearings, bushings, or other low-friction interfaces may also be included to support rotational components of the drive train and reduce friction during operation.

The actuator 404 may include an engagement member 413 extending from the housing 407. The engagement member 413 may be configured to allow a user to stabilize or grip the actuator 404 while operating the activator 410. For example, a user may engage the engagement member 413 with one or more fingers, such as the index and middle fingers, while actuating the activator 410 with the thumb.

The engagement member 413 may include a flange, collar, ring, finger rest, or one or more protrusions extending outward from the housing 407. In some configurations, the engagement member may extend circumferentially around at least a portion of the housing 407 to form a generally annular support surface. In other configurations, the engagement member may include one or more radially extending projections spaced around the housing to provide engagement surfaces for a user.

The engagement member 413 may be integrally formed with the housing 407 or may be provided as a separate component coupled to the housing. The engagement member may include textured surfaces, ridges, or ergonomic contours configured to improve grip and stability during actuation of the actuator 404.

In some examples, the actuator 404 may be configured as a disposable device intended for single use. Such disposable actuators may be manufactured from materials suitable for medical use and may be provided in a sterile condition. In other examples, the actuator 404 may be configured for multiple uses. A reusable actuator 404 may be manufactured from materials capable of being sterilized between uses, such as stainless steel, high-performance polymers, or other sterilizable materials.

Referring to FIG. 7, the plunger 405 may be detachably coupled to the actuator 404. The actuator 404 may include a ram 409 configured to engage and move the plunger 405. The ram 409 may include a coupling feature configured to mate with a plunger coupling feature 411 provided on the plunger 405. The ram coupling feature and the plunger coupling feature 411 may include a luer lock connector, a threaded connector and mating recess, a snap-fit connection, a press-fit interface, or another mechanical coupling mechanism. In some examples, the ram 409 may be configured to engage a variety of plungers having different diameters so that the actuator 404 may be used with syringes of different sizes. For example, the plunger 405 may be configured for use with syringes having capacities of approximately 5, 10, 20, 30, 50, or 60 cubic centimeters.

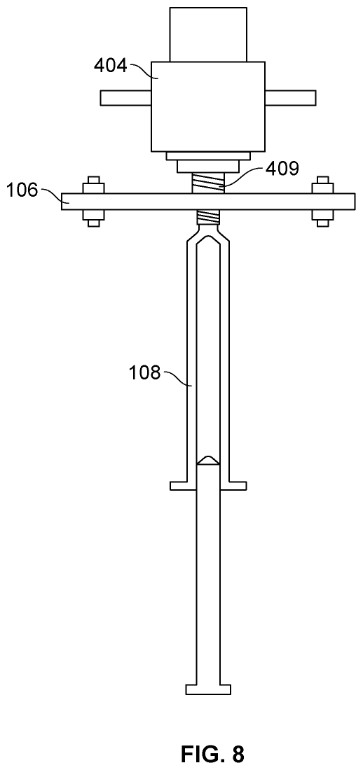

Referring to FIG. 8, the actuator 404 may alternatively be coupled directly to the processing module 106. In some examples, the ram 409 may be coupled to the hub 128 of the processing module 106 such that the ram 409 and the hub 128 are rotationally fixed relative to one another. The second syringe 108 may be coupled to the hub 128 as previously described. In such examples, the actuator 404, the processing module 106, and the second syringe 108 may form a rotationally coupled assembly. Rotation of the assembly about a common rotational axis may generate centrifugal forces that separate adipose tissue from oil or other biological components contained within the biological material. In some examples, rotation of the assembly may occur while biological material is transferred between syringes through the processing module 106, thereby allowing centrifugal separation and micronization to occur simultaneously.

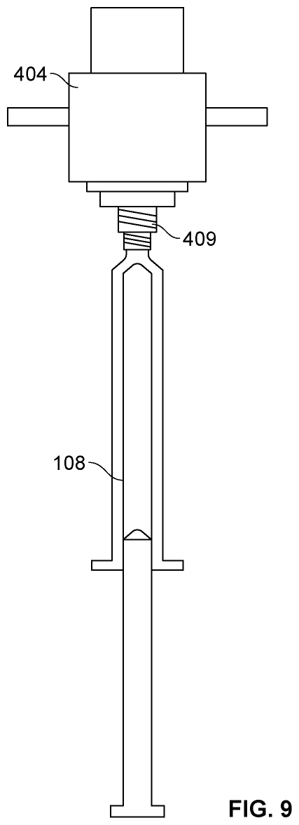

Referring to FIG. 9, the actuator 404 may be coupled directly to the second syringe 108. In this configuration, the actuator 404 may be used to rotate the second syringe 108 about a rotational axis extending generally along a longitudinal axis of the syringe. Rotation of the second syringe 108 may generate centrifugal forces to promote separation of adipose tissue from oil or other biological components prior to delivery of the processed adipose tissue to a patient.

The ram 409 may be coupled to the second syringe 108 such that the ram 409 and the second syringe 108 are rotationally fixed relative to one another. When coupled in this manner, rotational movement generated by the drive assembly of the actuator 404 may be transmitted to the second syringe 108 so that the syringe rotates together with the ram 409. This configuration may allow the actuator 404 to be used to perform an additional separation step after micronization and transfer of the biological material through the processing module.

The actuator 404 may be configured for use with the first syringe 102, the second syringe 108, or both syringes during operation of the processing assembly. For example, the actuator 404 may initially be coupled to the first syringe 102 to rotate the first syringe while biological material is transferred between the first syringe 102 and the second syringe 108 through the processing module 106. Rotation of the first syringe during transfer of the biological material may assist in separating adipose tissue from oil and other biological components while the biological material is being micronized within the processing module. After the biological material has been transferred to the second syringe 108, the actuator 404 may be detached from the first syringe 102 and coupled to the second syringe 108. The actuator 404 may then be used to rotate the second syringe 108 to further promote centrifugal separation of adipose tissue from oil prior to injection of the processed adipose tissue into a patient. In other configurations, the actuator 404 may be used with both syringes simultaneously, such as when the actuator is coupled to a component of the processing module 106 that transmits rotational motion to both syringes. This arrangement may allow the first syringe, the processing module, and the second syringe to rotate together as a rotational assembly during processing of the biological material.

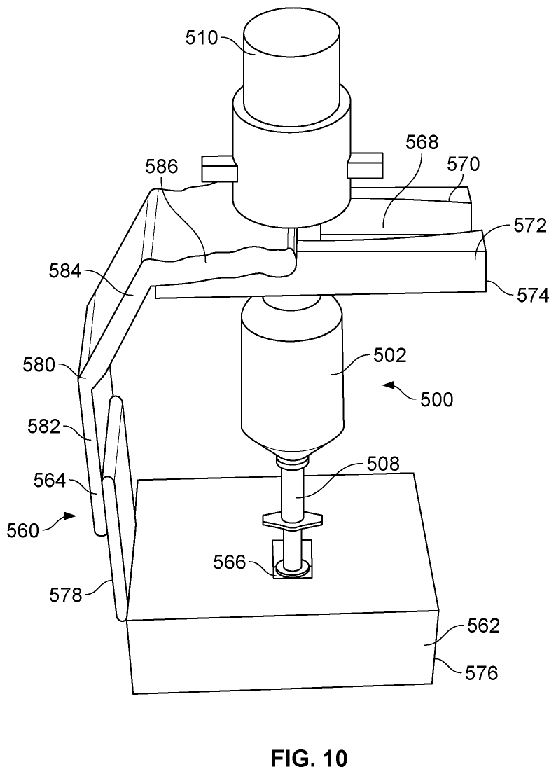

Now with reference to FIGS. 10 to 14, alternative constructions of a processing assembly or components thereof are illustrated. It should be appreciated that the processing assemblies and components thereof in FIGS. 10 to 14 are similar to the processing assembly 100 and only the differences between the processing assemblies and components thereof in FIGS. 10 to 14 and processing assembly 100 are discussed in detail herein. Further, like components are given a like reference number plus “500”; “600”; “700”; and “800” respectively.

Referring to FIG. 10, a support structure 560 may support the processing assembly 500 during operation. The support structure 560 may be configured to stabilize the processing assembly 500 while biological material is being processed. The support structure 560 may be configured to secure the processing assembly 500 in an upright position such that the first syringe 502 is above the second syringe 508. The support structure 560 may be configured to resist forces generated by the processing assembly 500 during rotation or actuation. For example, the support structure 560 may resist forces generated when a user actuates the activator 510 or when components of the processing assembly 500 rotate during operation. The support structure 560 may also be configured to permit rotation of one or more components of the processing assembly 500 while maintaining the processing assembly in a stable position.

The support structure 560 may allow a user to operate the processing assembly 500 using one hand while the processing assembly 500 is supported by the support structure 560. The support structure may stabilize the processing assembly during micronization, separation, or transfer of biological material between syringes. The support structure may also allow the processing assembly to be positioned on a table, tray, or other work surface during use.

The base 562 and the frame 564 may be formed as a unitary structure. Alternatively, the base 562 and the frame 564 may be separate components that are coupled together using adhesive, fasteners, welding, or other suitable attachment techniques. The base 562 and the frame 564 may be manufactured from a variety of materials including polymeric materials, metals, composites, or other rigid materials capable of supporting the processing assembly 500. The base 562 and the frame 564 may also be manufactured from different materials depending on structural requirements.

The base 562 may define a base coupling element 566 operable to engage at least one of the first and second syringes 502 and 508. The base coupling element 566 may include a recess, socket, or other receiving feature configured to receive at least a portion of the second syringe 508. For example, the recess may receive a distal portion of the syringe, a plunger end, or another portion of the syringe structure. In some configurations, the base coupling element 566 may be adjustable to accommodate syringes having different sizes or geometries. In other configurations, the support structure 560 may include one or more adapters configured to interface between the second syringe 508 and the base coupling element 566 so that the support structure can receive syringes of various sizes. The base coupling element 566 may be configured to receive syringes having different diameters or volumes, including syringes having volumes of approximately 1 cc, 5 cc, 10 cc, 20 cc, 30 cc, 50 cc, or 60 cc.

The frame 564 may be configured to engage at least one of the first syringe 502 or the second syringe 508. For example, the frame 564 may engage a portion of the first syringe 502 while the base 562 engages the second syringe 508 to secure the processing assembly 500 within the support structure 560. In this manner, the frame 564 and the base 562 may collectively define a receiving area configured to receive the processing assembly 500. The frame 564 may define a frame coupling element 568 configured to engage the first syringe 502. The frame coupling element 568 may include a recess, clamp, bracket, or other connector configured to engage the syringe. In some configurations, the frame coupling element 568 may include a first member 570 spaced from a second member 572 such that at least a portion of the first syringe 502 may be positioned between the first and second members 570 and 572. A frame end 574 may extend laterally further than a base end 576. This configuration may allow the first syringe 502 to engage the frame coupling element 568 before the remainder of the processing assembly 500 is positioned above the base 562, thereby facilitating placement of the processing assembly within the support structure.

The frame coupling element 568 may be movable relative to the base 562 to adjust a height of the receiving area defined by the support structure 560. For example, the frame 564 may be movable relative to the base 562. In some configurations, the frame 564 may be formed as a unitary structure. In other configurations, the frame 564 may include a first frame member 578 and a second frame member 580. The first and second frame members 578 and 580 may be movable relative to each other to adjust the height of the receiving area. The first and second frame members 578 and 580 may be detachably coupled using magnets, mechanical fasteners, hook-and-loop fasteners, or other coupling mechanisms. The first and second frame members 578 and 580 may be movable to discrete positions relative to each other. Alternatively, the frame members may be positioned at a desired location and secured using a locking mechanism. The locking mechanism may include a detent and recess arrangement, set screw, clamp, latch, buckle, or other locking structure. The frame 564 may therefore be adjustable to accommodate processing assemblies having different lengths or geometries.

The first frame member 578 may be coupled to the base 562. The first frame member 578 may be fixed to the base 562 using adhesive, mechanical fasteners, welding, or other attachment methods. The first frame member 578 may extend upwardly from an upper surface of the base 562. The first frame member 578 may be generally planar, although other configurations are possible. For example, the first frame member 578 may include bends, curves, or other structural features to improve strength or positioning of the processing assembly.

At least a portion of the second frame member 580 may overlap a portion of the first frame member 578. The second frame member 580 may include an elongate body that extends upward from the first frame member. The second frame member 580 may include one or more bends or angled sections. For example, the second frame member 580 may include a first section 582 and a second section 584 disposed at an angle relative to each other. The second section 584 may also be connected to a third section 586. The second and third sections 584 and 586 may be disposed at an angle relative to each other. The angled configuration of the sections may position the frame coupling element 568 at a desired location relative to the base 562.

The frame coupling element 568 may be coupled to the frame 564. For example, the frame coupling element 568 may be coupled to the third section 586 of the second frame member 580. The frame coupling element 568 may be detachably coupled to the frame 564 so that the position of the frame coupling element may be adjusted. In some configurations, the frame coupling element 568 may be repositionable along the frame 564 to permit lateral or longitudinal adjustment relative to the processing assembly 500. In other configurations, the frame coupling element 568 may be integrally formed with the frame 564.

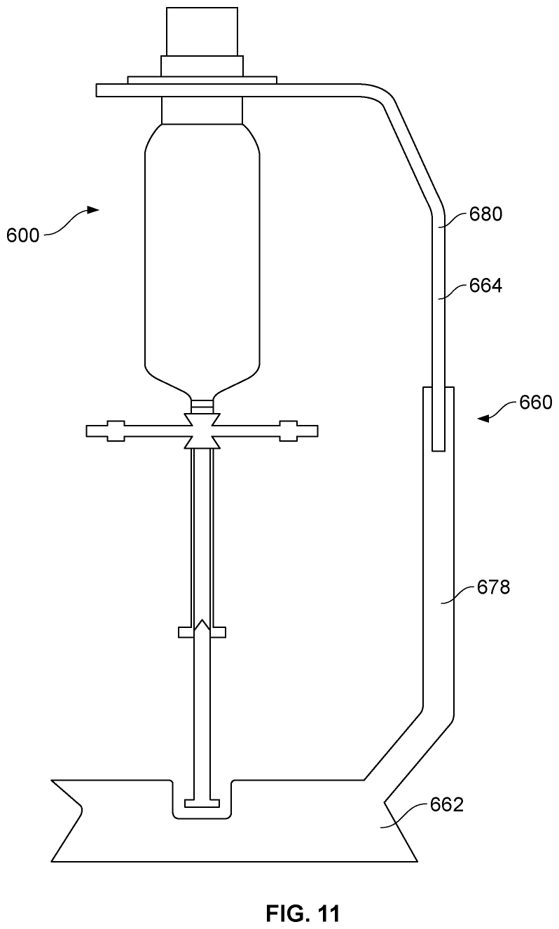

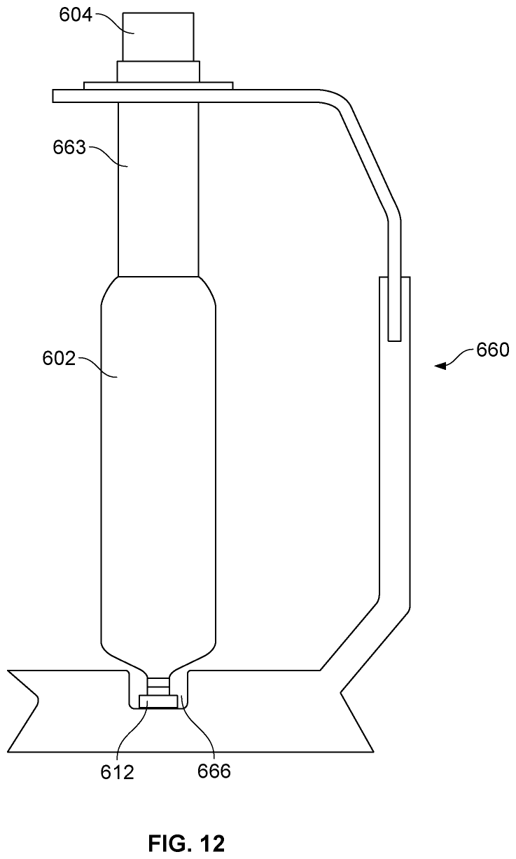

Referring to FIGS. 11 and 12, an alternative support structure 660 may be configured to support the processing assembly 600 during operation. The support structure 660 may include a base 662 and a frame 664 extending from the base 662. The frame 664 may include a first frame member 678 and a second frame member 680. The first and second frame members 678 and 680 may be movable relative to each other to adjust a height or position of a receiving area configured to receive the processing assembly 600.

One of the first and second frame members 678 and 680 may include a recess configured to receive a protrusion formed on the other of the first and second frame members. Engagement between the recess and the protrusion may allow the frame members to be positioned relative to each other while maintaining alignment of the frame 664. The recess and protrusion may further include a locking element configured to secure the first and second frame members 678 and 680 in a selected position. The locking element may include a ratchet mechanism, ball-and-detent mechanism, latch, spring-biased engagement member, or other mechanical locking system capable of holding the frame members in a fixed position relative to each other. In this manner, the support structure 660 may allow adjustment of the frame height to accommodate processing assemblies having different sizes or geometries.

The first and second frame members 678 and 680 may be selectively positioned relative to each other using a manually insertable positioning member. The positioning member may be configured as a pin, rod, pogo-pin, or similar elongate element that is receivable within aligned openings formed in the first and second frame members 678 and 680. The openings may be arranged in a series along at least one of the frame members such that the positioning member can be inserted at different locations to define discrete height positions of the frame 664. The positioning member may be tethered to at least one of the frame members 678 and 680. The positioning member may be tether via a flexible connector including a leash, cable, chain, or strap to prevent loss of the positioning member during use. Engagement of the positioning member with the aligned openings may maintain the relative position of the frame members under load. This arrangement may be similar to a pin-based height adjustment mechanism used in exercise equipment or adjustable support structures. In some implementations, the positioning member may include a retention feature such as a spring-loaded detent, clip, or enlarged head to resist unintended removal from the openings.

Referring to FIG. 12, the support structure 660 may alternatively support the first syringe 602 independently of the remainder of the processing assembly. In this configuration, the first syringe 602 may be coupled to the actuator 604 such that the actuator can drive movement of a plunger within the syringe. The actuator 604 may include a ram 663 configured to engage the plunger positioned within the first syringe 602. The ram 663 may be coupled to the plunger such that the ram and the plunger are rotationally fixed relative to each other. In this arrangement, movement of the ram 663 by the actuator 604 may cause corresponding movement of the plunger within the syringe 602. Rotational movement of the ram 663 caused by the actuator 604 may therefore be transferred to the first syringe 602 such that the two rotate together.

The ram 663 may be detachably coupled to the plunger of the first syringe 602. For example, the ram 663 may include a coupling feature configured to engage a corresponding plunger coupling feature. The coupling interface may include a threaded connection, Luer connector, snap-fit interface, press-fit interface, or other mechanical coupling capable of transmitting motion between the ram and the plunger.

The coupling element 612 of the first syringe 602 may be received within the base coupling element 666 of the base 662. Engagement between the coupling element 612 and the base coupling element 666 may stabilize the syringe during operation while permitting rotation of the syringe about a rotational axis. In this configuration, the support structure 660 may support the syringe and actuator assembly while the actuator 604 drives the plunger and rotates the syringe to facilitate separation of adipose tissue from oil or other biological components contained within the biological material. The actuator 604 may rotate the ram 663 and the syringe 602 while the ram 663 is in an extended position.

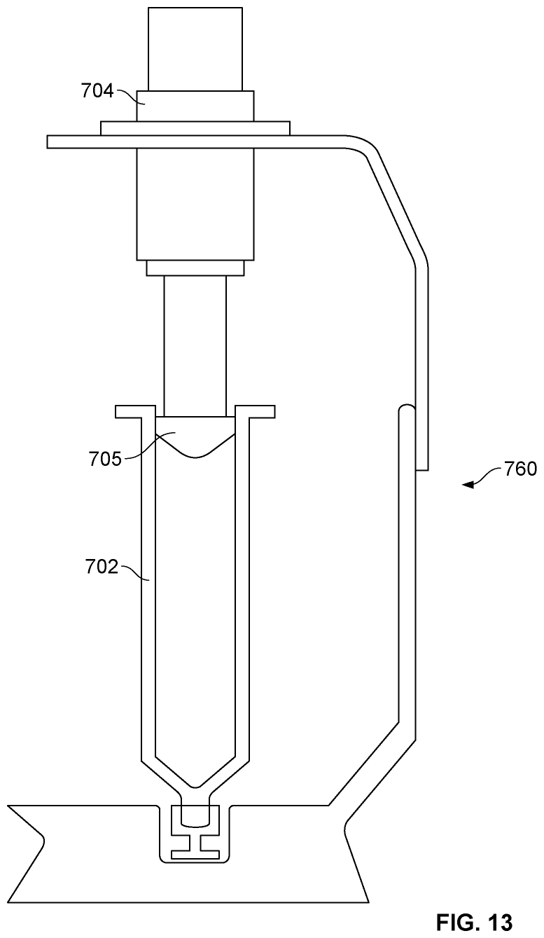

Referring to FIG. 13, the actuator 704 may be detachably coupled with the first syringe 702. The plunger 705 may include a ram that connects a head of the plunger to the actuator 704. The plunger 705 may be removably coupled to the actuator 704. The plunger 705 may be removably positioned within the barrel of the first syringe 702. In some examples, the actuator 704 is fixed to the support structure 760. In other examples, the actuator 704 is detachably coupled to the support structure 760. The actuator 704 may be detached from the syringe 702 while the syringe 702 is coupled to the support structure 760. The actuator 704 may be detached from the support structure 760 while the syringe 702 is coupled to the support structure 760.

Referring to FIG. 13, the actuator 704 may be detachably coupled with the first syringe 702. The first syringe 702 may include a barrel configured to receive biological material and a plunger 705 positioned within the barrel. The plunger 705 may include a head portion configured to form a fluid seal with an inner wall of the barrel and a ram extending from the head portion toward the actuator 704. The ram may connect the head of the plunger to the actuator 704 so that movement of the actuator causes corresponding movement of the plunger within the barrel.

The ram of the plunger 705 may be removably coupled to the actuator 704. For example, the ram may include a coupling feature configured to engage a corresponding coupling feature of the actuator. The coupling interface may include a threaded connection, snap-fit connection, press-fit connection, bayonet connection, or other mechanical coupling configured to transmit motion between the actuator and the plunger. The plunger 705 may also be removably positioned within the barrel of the first syringe 702 so that the plunger may be replaced or removed when desired.

In some configurations, the actuator 704 may be fixed relative to the support structure 760. For example, the actuator 704 may be mounted to the support structure using fasteners, adhesive, welding, or other attachment mechanisms. In other configurations, the actuator 704 may be detachably coupled to the support structure 760 so that the actuator can be removed from the support structure when desired.

The actuator 704 may be detached from the first syringe 702 while the syringe 702 remains coupled to the support structure 760. Likewise, the actuator 704 may be detached from the support structure 760 while the syringe 702 remains supported by the support structure 760. This arrangement may allow the actuator to be selectively coupled to or removed from the syringe without removing the syringe from the support structure, thereby facilitating replacement of components or reconfiguration of the processing assembly.

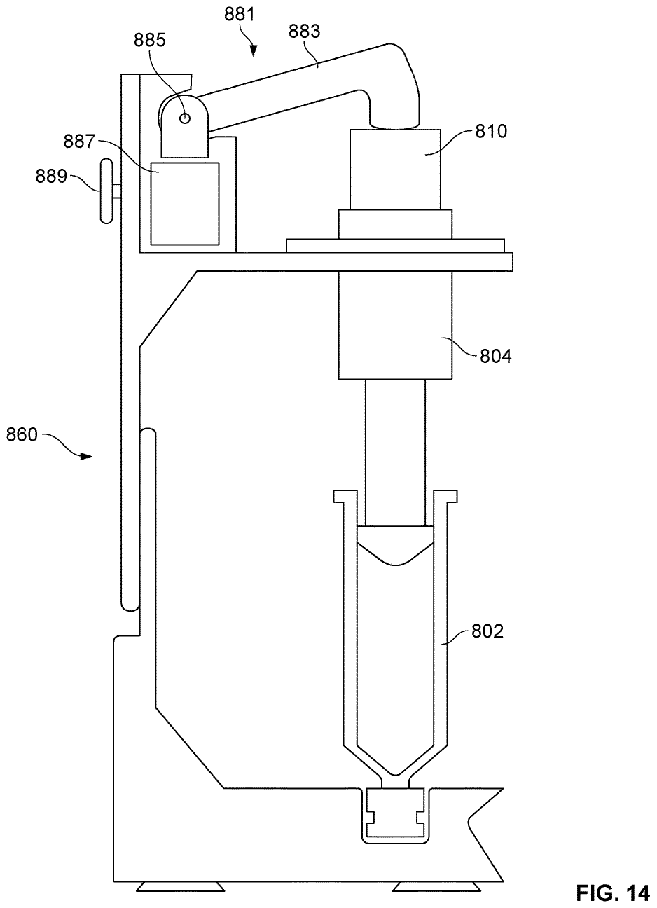

Referring to FIG. 14, a support structure 860 may be configured to support a syringe 802 during operation. The support structure 860 may alternatively support additional components of a processing assembly, or the entire processing assembly, as described above. The support structure 860 may include an automated actuation assembly 881 configured to actuate the syringe 802. The automated actuation assembly 881 may be configured to rotate a plunger 805 that is within a barrel of the syringe 802. The plunger 805 may be rotationally fixed relative to the barrel of the syringe 802 such that the plunger 805 and syringe 802 rotate together. The automated actuation assembly 881 may be configured to engage an activator 810 of an actuator 804 that is coupled to the plunger 805.

The automated actuation assembly 881 may include a motor 887 or another drive element configured to move the arm 883. The motor 887 may be coupled directly to the arm 883 or may be coupled to the arm through a drive train including gears, cams, linkages, or other motion-transmitting elements. Operation of the motor 887 may therefore cause the arm 883 to pivot about the axis 885 and press the activator 810. The motor 887 may be powered by an internal power source, such as one or more batteries, or may receive power from an external power source such as a wall outlet, power supply, or charging interface.

Movement of the arm 883 may be generated by a variety of motion conversion mechanisms, including cams, eccentrics, crank mechanisms, gears, linkages, solenoids, or other mechanical or electromechanical drive systems configured to periodically actuate the manually operable element. In some examples, the actuation assembly 881 may include a motor 887 to move the arm 883. The motor 887 may be coupled directly to the arm 883 or may be coupled by gearing or linkage. The motor 887 may be coupled to a power source such as a battery or a wall outlet.

The arm 883 may be configured to repeatedly press the activator 810 during operation of the automated actuation assembly 881. Repeated actuation of the activator 810 may cause the actuator 804 to rotate the syringe 802 at a desired rotational speed. The rotational speed of the assembly may be selected to generate sufficient centrifugal force to promote separation of the biological components.

The automated actuation assembly 881 may include a speed selector 889 configured to control the rate at which the arm 883 presses the activator 810. The speed selector 889 may include a potentiometer, dial, slider, push-button control, or other user interface configured to adjust operation of the motor 887. The automated actuation assembly 881 may also include a timer configured to deactivate the actuation assembly after a selected time interval. In some configurations, the automated actuation assembly 881 may be fixed to the support structure 860. In other configurations, the automated actuation assembly may be detachably coupled to the support structure. At least one of the syringe 802 or the actuator 804 may be decoupled from the support structure 860 while the automated actuation assembly 881 remains coupled to the support structure.

The automated actuation assembly 881 may be used with a variety of manually actuated devices. Although the automated actuation assembly 881 is illustrated as operating the activator 810 of the actuator 804 coupled to the syringe 802, the automated actuation assembly may alternatively be configured to actuate other manually operable mechanisms. For example, the automated actuation assembly 881 may be configured to repeatedly actuate push-buttons, plungers, triggers, or other manually actuated elements of medical devices or laboratory devices. In this manner, the automated actuation assembly may convert a manually actuated device into an automatically operated device while the device remains supported by the support structure 860.

Aspects of the present disclosure are not limited to the exemplary structural details and component arrangements described in this description and shown in the accompanying drawings. Many aspects of this disclosure may be applicable to other aspects and/or capable of being practiced or carried out in various variants of use, including the examples described herein. It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Several different reference axes are described, including a lateral axis X-X, a longitudinal axis, Y-Y, and/or a vertical axis Z-Z oriented about a datum to form a three-dimensional Cartesian coordinate system. Relevant arrangements may be described in relation to different reference axes. For example, the longitudinal axis may be non-parallel with the lateral axis in some perspectives, meaning that one axis extends across the other; and the vertical axis may be non-parallel with the lateral and longitudinal axis in some perspectives, meaning that all three axes extend across one another. Relative terms such as “long” and “elongated” may describe any aspect having a length along one reference axis (e.g., the longitudinal or vertical axis) that is longer in relation to a width along a non-parallel reference axis (e.g., the lateral axis). As utilized herein, these terms are provided for convenience and not intended to limit this disclosure unless claimed.

The terms “proximal” and “distal” may be used to describe some structures in relation to a reference axis. As utilized herein, these terms are provided for convenience to orient the reader and not intended to limit this disclosure unless claimed. Movements and forces may be similarly described in relation to any reference axis. Here again, descriptions of certain movements and forces in relation to certain reference axes are provided to help explain certain inventive aspects by way of example and are not intended to limit this disclosure unless claimed.

The words “right”, “left”, “lower” and “upper” designate directions in the drawings to which reference is made. The words “inwardly” and “outwardly” refer to directions toward and away from, respectively, the geometric center of the processing assembly.

Unless specifically set forth herein, the terms “a”, “an” and “the” are not limited to one element but instead should be read as meaning “at least one”. Inclusive terms such as “comprises,” “comprising,” “includes,” “including,” and variations thereof, are intended to cover a non-exclusive inclusion, such that aspects of any apparatus, kit, method, and system described herein, or element(s) thereof described as comprising a list of elements does not include only those elements but may include other elements not expressly listed and/or inherent thereto. Unless stated otherwise, the term “exemplary” means “example” rather than “ideal.” Various terms of approximation may be used, including “approximately” and “generally.” Approximately means “roughly” or within 10% of a stated number or outcome and generally means “usually” or more than a 50% probability of a stated number or outcome.

Connective terms such as “coupled to,” “coupled with,” and “coupling” are intended to generically describe a structural connection between two or more elements. Some structural connections may be “fixedly coupled” so that the connected elements are generally non-rotatable or non-movable relative to one another, as when the elements are formed together (e.g., cast, bolted, and/or welded) and cannot be rotated independently without deflecting relative to one another or being damaged. Other structural connections may be “rotatably or movably coupled” so that the connected elements are coupled together to permit movements relative to one another, as when the elements are pinned together (e.g., with any type of rotating, sliding, and/or telescoping connection) and can be rotated or moved freely and independently without damage. Unless stated otherwise, these exemplary connective terms and their modifiers may comprise any such variations.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined.

To the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

I/We claim:

1. A biological tissue processing assembly comprising:
a first syringe configured to contain biological material;
a second syringe fluidly connectable to the first syringe to receive biological material from the first syringe;
a processing device fluidly coupled to the first syringe and the second syringe, the processing device comprising a body with an aperture such that biological material passes through the aperture as the biological material passes between the first syringe and the second syringe; and
an actuator configured to rotate at least one of the first syringe, the second syringe, and the processing device to generate centrifugal force that separates components of the biological material.

2. The assembly of claim 1, wherein the actuator rotates the first syringe and the second syringe simultaneously.

3. The assembly of claim 2, wherein the actuator rotates first syringe, the second syringe, and the processing device simultaneously.

4. The assembly of claim 2, wherein the actuator rotates the first and second syringes about a common axis.

5. The assembly of claim 1, wherein the aperture is one of a plurality of apertures and the body includes a plurality of transfer arms extending outward from a central portion of the body, each transfer arm including at least one aperture.

6. The assembly of claim 5, wherein the transfer arms are circumferentially spaced about the central portion.

7. The assembly of claim 1, wherein the aperture is one of a plurality of apertures and each aperture mechanically reduces a particle size of the biological material passing through the processing device.

8. The assembly of claim 7, wherein the apertures have progressively decreasing diameters.

9. The assembly of claim 1, wherein the actuator comprises a manually activated device that converts linear motion to rotational motion.

10. The assembly of claim 1, wherein the actuator is detachably coupled to the first syringe.

11. The assembly of claim 1, wherein the processing device includes a filter.

12. The assembly of claim 1, wherein the aperture is one of a plurality of apertures and each of the first and second syringes are detachably coupled to the same aperture when biological material passes from the first syringe to the second syringe.

13. The assembly of claim 1, wherein the processing device further comprises an absorbent material configured to absorb oil separated from adipose tissue.

14. A biological tissue processing assembly comprising:
a base;
a frame extending from the base; the frame configured to secure a syringe containing biological material; and
an actuator coupled to the frame and configured to engage and rotate the syringe to separate adipose tissue from oil or other biological components.

15. The assembly of claim 14, further comprising:
a first syringe configured to contain biological material, the first syringe coupleable to the frame;
a second syringe fluidly connectable to the first syringe to receive biological material from the first syringe, the second syringe coupleable to the base; and
a processing device fluidly coupled to the first syringe and the second syringe, the processing device having a body with an aperture such that biological material passes through the aperture as the biological material passes between the first syringe and the second syringe.

16. The assembly of claim 14, wherein the actuator includes a push button and linear motion of the push button is converted into rotational motion of the syringe.

17. The assembly of claim 16, further comprising an arm movably coupled to the frame, the arm configured to move the push button linearly to cause rotational motion of the syringe.

18. The assembly of claim 17, further comprising a motor coupled to the arm to move the arm relative to the frame.

19. The assembly of claim 14, wherein the frame and the base define a receiving area for the syringe,
wherein at least a portion of the frame is moveable relative to the base from a first position to a second position, and
wherein and the receiving area has a first height when the frame is in the first position and a second height different from the first height when the frame is in the second position 20. The assembly of claim 14, wherein at least one of the frame and the base includes a locking mechanism configured to hold the syringe in a fixed position during actuation.

21. The assembly of claim 14, wherein the base includes a recess to receive at least a portion of the syringe to stabilize the syringe device during rotation.

22. A method of processing adipose tissue comprising:
placing adipose tissue into a first syringe;
coupling the first syringe to a processing device including an aperture;
coupling a second syringe to the processing device;
transferring the adipose tissue from the first syringe through the aperture and to the second syringe; and
rotating at least one of the processing device, the first syringe, and the second syringe to generate centrifugal force that separates adipose tissue from oil or other biological components.

23. The method of claim 22, wherein the aperture is one of a plurality of apertures and the method further comprises sequentially coupling the first and second syringes to the apertures and passing the adipose tissue through the apertures to micronize the adipose tissue.

24. The method of claim 22, further comprising:
coupling the first syringe to an actuator prior to the rotating step; and
activating the actuator to rotate at least one of the processing device, the first syringe, and the second syringe.

25. The method of claim 24, further comprising:
decoupling the first syringe from the actuator;
coupling the second syringe to the actuator; and
activating the actuator to rotate the second syringe to generate centrifugal force that further separates adipose tissue from oil or other biological components.

26. The method of claim 22, wherein the aperture is a first aperture and coupling the first and second syringes to the processing device includes coupling the first and second syringes to the processing device in a first orientation such that the first and second syringes are in fluid communication with the first aperture, and
wherein the processing device includes a second aperture and the method further comprises:
decoupling the first and second syringes from the processing device;
coupling the first and second syringes to the processing device in a second orientation such that the first and second syringes are in fluid communication with the second aperture; and
transferring the adipose tissue from the second syringe through the second aperture to the first syringe.

27. The method of claim 26, wherein the rotating step includes rotating the rotating the processing device about a first axis when the first and second syringes are in the first orientation and the method further comprises:
rotating the processing device about a second axis when the first and second syringes are in the second orientation.

28. The method of claim 26, wherein decoupling the first syringe from the actuator includes decoupling the first syringe and the processing device from the actuator.

29. The method of claim 24, further comprising:
decoupling the first syringe from the actuator;
coupling the processing device to the actuator; and
activating the actuator to rotate the processing device and the second syringe.

30. The method of claim 22, wherein the rotating step includes simultaneously rotating each of the processing device, the first syringe, and the second syringe about a common axis.

31. The method of claim 22, further comprising reinjecting processed adipose tissue into a patient.

32. The method of claim 31, wherein the reinjecting step includes reinjecting the processed adipose tissue from the second syringe into the patient.

33. The method of claim 22, further comprising:
transferring the adipose tissue from the second syringe through the aperture to the first syringe.

34. The method of claim 33, wherein the first and second syringes remain coupled to the processing device as the adipose tissue is transferred through the aperture from the first syringe to the second syringe and from the second syringe to the first syringe.
